(19) United States
(12) Patent Application Publication
(10) Pub. No.: US 2025/0186697 A1
(43) Pub. Date: Jun. 12, 2025

(54) WORKPIECE CARRIER FOR AN INJECTION DEVICE

(71) Applicant: Sanofi, PARIS (FR)

(72) Inventors: Andre BENISCHKE, Frankfurt am Main (DE); Holger DIEFENBACH, Frankfurt am Main (DE); Julian KERSTING, Rüsselsheim (DE); Benjamin MADSACK, Frankfurt am Main (DE); Peter NOBER, Rüsselsheim (DE); Nikolai URBAN, Rüsselsheim (DE)

(21) Appl. No.: 18/843,669

(22) PCT Filed: Mar. 9, 2023
(86) PCT No.: PCT/EP2023/055989

§ 371 Date: Sep. 3, 2024

(30) Foreign Application Priority Data
Mar. 11, 2022 (EP) 22315058.2

(51) Int. Cl. A61M 5/20

(52) U.S. Cl. CPC A61M 5/20; A61M 2209/082

(57) ABSTRACT

Workpiece Carrier for an Injection Device The present disclosure relates to a workpiece carrier (30) for holding a device component (100) of a drug delivery device (1) in an automated production line, the workpiece carrier (30) comprising:—a body (31) extending in a longitudinal direction (z), the body (31) defining a receiving space (33) sized to receive at least a portion of the device component (100),—a locking mechanism (50; 150) comprising a first locking member (51;151) movable relative to the body (31) between a locking position and an unlocking position,—wherein the first locking member (51; 151) comprises a receiving section (56) and an engaging section (76; 176) separated from the receiving section (56),—wherein the receiving section (56) adjoining the receiving space (33) comprises an inside face (60) with a first contact section (62) facing radially inwardly towards the receiving space (33) to make contact with the device component (100) upon insertion of the device component (100) into the receiving space (33) along the longitudinal direction (z) to thereby induce a movement of the first locking member (51; 151) into the locking position.

### FIELD

The present disclosure relates to a workpiece carrier, in particular configured as an assembly nest for receiving and holding a device component of a drug delivery device and/or for receiving and holding a drug delivery device, typically in an automated production line. In a further aspect the present disclosure relates to a method of holding a device component, e.g. a subassembly of a pen-type injector, such as an autoinjector in an automated production line by using a workpiece carrier.

### BACKGROUND

Drug delivery devices allowing for single or multiple dosing of a required dosage of a liquid medicinal product and further providing administration of such liquid drug to a patient, are as such well known in the prior art. Generally, such devices have substantially the same purpose as that of an ordinary syringe. Typically, a medicinal product to be administered is provided in a medicament container having a moveable piston or bung mechanically interacting with a piston rod of a drive mechanism of the drug delivery device. By applying thrust to the piston, a certain and pre-defined amount of the medicinal fluid can be expelled from the medicament container. The medicament container may comprise a syringe or cartridge.

Manufacturing and final assembling of such drug delivery devices is implemented in a mass production process. In a typical final assembly scenario two sub-assemblies have to be assembled with each other. For instance, a first sub-assembly comprises the medicament container and the second sub-assembly comprises a housing or body adapted to receive the drive mechanism. With further examples, the drug delivery device or injection device comprises a housing, which in the course of device manufacturing has to be kept and held in particular orientation, e.g. for automatically attaching a label thereto. Since the final assembly is conducted almost entirely automatically, the subassemblies, device components and/or injection devices have to be provided in a well-defined and ordered way. Hence, the subassemblies have to be correctly oriented and disposed on a respective support structure, such as an assembly nest.

A workpiece carrier or an assembly nest should also provide a rather stable, tilt-free holding or supporting of a device preassembly of an injection device. With some automated production lines it is required to keep or to hold the device subassembly in an upright orientation, e.g. with a long axis of the subassembly pointing in vertical direction. In order to hold or to support a subassembly in a tilt-free and stable manner the nest assembly has to be particularly dedicated or configured for each type of drug delivery device or subassembly. Hence, for manufacturing a large number of different drug delivery devices, e.g. a drug delivery device of a first type and a drug delivery device of a second type different nest assemblies may be required.

It is hence an object to provide an improved assembly nest that is universally suitable for holding not only one specific but at least two differently configured or differently-shaped subassemblies or device components of a drug delivery device. The nest assembly should provide a rather easy and failure safe handling. It should provide a tilt-free and stable holding of the subassembly during and for an automated assembly or production process. The nest assembly should further minimize the risk of negative product influences, damages and malfunctions of the drug delivery device during automated production thereof

### SUMMARY

In one aspect there is provided a workpiece carrier for holding a device component of a drug delivery device in or for an automated production line. With some examples, the workpiece carrier may be configured to receive and to hold a housing or housing component of the drug delivery device and hence the entire drug delivery device. The workpiece carrier comprises a body extending in a longitudinal direction. The body defines a receiving space or accommodating space, which is sized to receive at least a portion of the device component of the drug delivery device. The workpiece carrier further comprises a locking mechanism. The locking mechanism comprises a first locking member. The first locking member is movable relative to the body between a locking position and an unlocking position. The first locking member comprises a receiving section and an engaging section. The receiving section and the engaging section are separated from each other. The receiving section adjoining the receiving space comprises an inside face with a first contact section. The first contact section faces radially inwardly towards the receiving space to make contact with the device component when the device component is inserted into the receiving space along the longitudinal direction to thereby induce a movement of the first locking member into the locking position.

The workpiece carrier, which may be implemented as an assembly nest for holding a device component, e.g. a subassembly of a drug delivery device, a housing component or the entirety of the drug delivery device, provides a kind of a self-locking or automated locking when the device component is inserted into the receiving space along the longitudinal direction. Typically, and when the device component, which may be of a sleeve-like or tubular shape, is inserted in a longitudinal distal direction into the e.g. sleeve-shaped receiving space the device component, e.g. a distal portion or distal end thereof may at some stage start to mechanically engage with the receiving section of the first locking member. The mechanical contact between the device component and the receiving section, in particular the mechanical contact between the device component and the first contact section of the receiving section induces a movement of the first locking member towards and into the locking position.

Hence, by inserting the device component into the receiving space the mutual mechanical contact between the device component and the first contact section leads to a movement of the first locking member towards and into the locking position. Moving of the first locking member into the locking position brings the engaging section of the first locking member in mechanical engagement with another portion of the device component. Typically, the engaging section may engage with a complementary shaped counter engaging section of the device component so as to fix the device component inside the receiving space when reaching a final assembly position inside the receiving space.

Typically, a motion of the first locking member from the unlocking position towards and into the locking position is entirely induced by the insert motion of the device component into the receiving space of the body.

An outside surface of the device component may engage, e.g. abut with or slide along the first contact section of the inside face of the receiving section of the first locking member. In this way, there can be induced a rather smooth and gradual movement of the locking member towards the locking position. The body of the workpiece carrier may be of tubular shape. With some examples the body comprises a tubular-shaped side wall and a hollow interior forming or constituting the receiving space for the device component.

Towards the distal end the receiving space may comprise a bottom face. The receiving space may be delimited or closed by the bottom face. The first locking member is typically movable with regards to a radial direction. Here and in the present context the radial direction may denote a direction that is perpendicular to the longitudinal direction. With the body comprising a cylindrical shape the radial direction extends towards or away from the radial center of the cylindrical sidewall of the body. Likewise, and when the body is of rectangular or for polygonal shape, the radial direction may extend along a surface normal of the sidewall of the body.

The first contact section faces radially inwardly. Typically, a surface normal of the first contact section of the inside face of the receiving section of the first locking member may slightly deviate from the strict geometric radial direction. Here, an angle extending by 15° to 30° or even up to 45° from a strict radial direction may be still regarded as substantially facing radially inwardly. Of course, and since the first contact section makes contact with a portion of the device component it is subject to a movement and may therefore change its orientation slightly.

Typically, the body comprises or provides a hollow interior comprising a cross-section that is larger than an outer cross-section of the device component. The movable first locking member may be configured to move radially inwardly, typically with its engaging section so as to reduce the free space between opposing inside facing sidewall sections of the body in order to hold or keep the device component in a tilt-free manner inside the receiving space.

With some examples the first locking member and hence the locking mechanism may serve to clamp the device component inside the receiving space, typically in radial direction, i.e. perpendicular to the longitudinal direction of the body.

With some examples the first contact section extends substantially parallel to the longitudinal direction as the first locking member is in a locking position. When in an unlocking position the first contact section may be oriented slightly offset relative to the longitudinal direction. Here, the first contact section may taper towards the distal longitudinal direction, i.e. it may reduce the inside diameter or inside cross-section of the preceding space as seen from the proximal towards the distal longitudinal direction. In this way, the first contact section may provide a kind of a beveled section that gets in contact with e.g. a distal end section of the device component upon insertion of the device component into the receiving space in longitudinal distal direction.

The initially beveled or oblique orientation of the first contact section helps to make a sliding contact, e.g. with a distal end of the device component upon insertion of the device component into the receiving space. As the device component makes contact with the first contact section and as the device component is moved further in distal direction towards a final assembly position it serves to urge the first contact section radially outwardly, thereby inducing a respective movement of the first locking member towards and into the locking position.

In this way and as the device component is moved any further in distal direction into the receiving space the first locking member continues to move towards the locking position until the device component has reached a distal end position inside the receiving space. When reaching the end position or final assembly position inside the receiving space the first locking member has also reached the locking position. Typically, and when arriving in the locking position the engaging section of the locking member engages with a complementary shaped counter engaging section of the device component, thereby clamping or holding the device component in a rather tilt free and stable manner on the workpiece carrier, e.g. inside the receiving space.

According to a further example the first locking member comprises a locking arm. The locking arm is pivotally attached to the body between the locking position and the unlocking position. Hence, the first locking member, in particular its locking arm can be pivoted from a locking position towards an unlocking position and vice versa. When in the unlocking position the receiving space is configured to receive the device component therein. After or during insertion of the device component the locking arm and hence the first locking member is pivoted or swiveled into the locking position. When or upon arriving in a final assembly position the locking arm and hence the first locking member gets into the locking position, thereby securing and/or fastening the device component to the workpiece carrier.

Locking the device components to the workpiece carrier may include a clamping of the device component inside the receiving space so as to prevent any tilting or the like motion of the device component relative to the body of the workpiece carrier. Typically, the longitudinal device component can be kept in an upright configuration inside the receiving space.

Typically, the pivoting axis, according to which the locking arm can be pivoted relative to the body extends in a horizontal direction or transverse direction, i.e. perpendicular to the longitudinal direction of the body. With some examples the pivoting axis or hinge axis is located inside a sidewall structure or sidewall of the body. It may extend in a circumferential or tangential direction with regards to an outer circumference of the tubular shaped body. This way, and by pivoting the locking arm between the locking position and the unlocking position the radial position of the receiving section and the radial position of the engaging section of the first locking member are subject to predefined variations.

With some examples and when the first locking member, e.g. the locking arm thereof substantially extends in longitudinal direction and when the receiving section and the engaging section are located at or near opposite longitudinal ends of the first locking member a radially outwardly directed motion of the receiving section of the locking member as induced by the insertion of the device component into the receiving space leads to a respective radially inwardly directed motion of the engaging section. With other words, the locking arm provides a kind of a lever by way of which a motion of the receiving section along or into a first direction can be transferred into a respective motion of the engaging section into a respective opposite direction.

According to a further example the receiving section and the engaging section of the first locking member are located on opposite sides of a hinge axis. This way, a radially outwardly directed movement of the receiving section inducible by insertion of the device component into the receiving space causes a radially inwardly directed movement of the engaging section.

The radially inwardly directed movement of the engaging section may lead to a radially inwardly directed clamping of the device component inside the receiving space or relative to the body. In this way, a rather tilt-free fastening and/or holding of the device component on or in the workpiece carrier can be provided.

Typically, the first locking member, and hence its locking arm is of rather stiff structure. In this way a radially outwardly directed movement of the receiving section as induced by longitudinal insertion of the device component inside the receiving space leads to a respective radially inwardly directed movement of the engaging section.

Typically, it is the distal end, e.g. a distal end face or a distally located side wall section of the device component that is configured to engage with the receiving section of the first locking member so as to induce the radially outwardly directed movement thereof. With typical examples the engaging section is located at a longitudinal distance from the receiving section. In this way and when the first locking member is moved or pivoted towards and into the locking position the engaging section engages with another portion of the device component that is located at a proximal longitudinal distance from the distal end of the device component.

In this way, there can be provided a twofold support and fastening of the device component inside the receiving space. The distal end section of the device component may make contact with the receiving section of the first locking member and the further portion of the device component may get in contact with the engaging section of the first locking member.

According to a further example the first locking member extends in the longitudinal direction. The receiving and the engaging sections are separated along the longitudinal direction. With some examples the engaging section is located at a proximal end of the first locking member or the locking arm thereof. The receiving section is located at a distal end of the first locking member, e.g. at the distal end of the locking arm.

The pivot axis or hinge axis of the first locking member may be located substantially midway between the opposite longitudinal end sections of the first locking member. With some examples the hinge axis may be located closer to one of the receiving section and the engaging section compared to the other one of the receiving section and the engaging section. In this way, a kind of a leverage effect can be provided. When the pivot axis or hinge axis of the locking arm or of the first locking member is located nearer to the receiving section than to the engaging section a comparatively small radially directed movement of the receiving section induces a comparatively large or at least a larger radially directed movement of the engaging section.

Vice versa and when the hinge axis is located closer to the engaging section than to the receiving section a comparatively large movement of the engaging section is transferred into a smaller movement of the engaging section. According to such a leverage effect, respective forces for inducing a movement of the first locking member and for engaging the engaging section of the first locking member with the device component are transferred across the locking arm accordingly.

With a further example the inside face of the receiving section comprises a first beveled section adjoining longitudinally the first contact section. The first contact section may extend substantially along the longitudinal direction, i.e. it may extend substantially parallel to the elongation of the first locking member, e.g. parallel to the longitudinal extension of the respective locking arm. The first beveled section is oriented obliquely relative to the first contact section. The first beveled section is typically located at a proximal distance from the first contact section. The first beveled section extends radially inwardly as seen towards the distal direction, hence towards a bottom of the receiving space.

In this way the first beveled section reduces the inside cross-section of the receiving space as seen from the proximal towards the distal direction, e.g. as seen along the insert direction for the device component. When inserting the device component into the receiving space in longitudinal distal direction a distal end section or a distally facing side wall structure of the device component starts to make contact with the first beveled section. By moving further into the receiving space in distal direction the device component then starts to slide along the first beveled section, thereby inducing a radially outwardly directed movement of the receiving section due to the beveled and hence oblique shape of the first beveled section.

Typically, and when reaching a final assembly position the respective contact face of the device component engages the first contact section of the receiving section of the first locking member. When reaching the locking position the first contact section of the receiving section of the first locking member may be complementary shaped to a respective counter contact face on the outside surface of the device component. In this way there can be provided a rather stable and well-defined mechanical engagement between the device component and the first contact section of the first locking member.

According to a further example the inside face of the receiving section comprises a second contact section facing radially inwardly towards the receiving space to make contact with their device component upon insertion into the receiving space. Typically, the first and second contact sections are provided at different radial positions. They may be located radially offset on the inside face of the receiving section of the first locking member. The first and second contact sections may be configured to engage with different types of device components, that may e.g. belong to different types of drug delivery devices.

Typically, the first receiving section may be configured and shaped to engage with a first device component. The second contact section may be configured to engage with a second device component. First and second device components may belong to different types, e.g. to a first and a second drug delivery device, respectively.

In this way, the workpiece carrier can be universally used for different or numerous types of device components. It may provide a stable and tilt-free holding of a first device component when the first contact section engages a respective first counter engaging section on the outside surface of the first device component. The second contact section of the inside face of the first locking member engages with a respective counter contact face on the outside surface of a second device component.

Typically, and according to a further example the second contact section is located distally from the first contact section.

With further examples the second contact section is located radially inwardly from the first contact section on the inside face of the first locking member. In this way, the inner diameter or inside dimensions of the receiving space in the region of the second contact section is smaller than the inner diameter or inside free space of the receiving space in the region of the first contact section. Accordingly, the first contact section is configured to engage with a device component comprising a first outer dimension. The second contact section is configured to engage with a second device component comprising a second outer dimension. Typically, the second outer dimension of the second device component is somewhat smaller than the first outer dimension of the first device component.

With some examples the first contact section defines or adjoins a longitudinal stop for a first device component. Hence, when a first counter contact surface of the first device component engages the first contact section of the first locking member there is also reached a distal end stop by way of which a longitudinal insertion of the device component into the receiving space is delimited or blocked.

It is then only a second device component comprising a respective counter contact section or counter contact surface featuring smaller outside dimensions compared to the first counter contact section of the first device component. The counter contact section of the second device component will be enabled to slide past the first contact section so as to engage with the second contact section, which is located distally from the first contact section.

According to a further example the inside face of the receiving section comprises a second beveled section adjoining longitudinally the first contact section and adjoining longitudinally the second contact section. With some examples and as seen from a proximal end towards a distal direction the inside face of the receiving section of the first locking member comprises the first beveled section adjoining in distal direction the first contact section. The first contact section then adjoins a longitudinally and distally the second beveled section and the second beveled section adjoins in longitudinal distal direction the second contact section.

Here, a proximal end of the second beveled section extending proximally into the first contact section may form or constitute a first longitudinal stop or distal stop for a first device component.

This way and when the first device component is inserted into the workpiece carrier a counter contact section of the first device component may suitably engage with the first contact section. A further distally directed movement of the first device component is then blocked by the radially narrowing and hence radially inwardly extending second beveled section.

According to a further example the workpiece carrier comprises at least a first counter stop on the body to engage with an outside facing outer stop of the first locking member. This way, a movement of the first locking member towards any of the locking position and the unlocking position can be delimited. When reaching the locking position e.g. an outer stop of the first locking member may engage a complementary shaped counter stop as provided on the body.

Here, the outer stop of the first locking member may be provided on an outside facing surface of the receiving section and/or at the distal end of the first locking member. Accordingly, another outside facing outer stop as provided on a proximal section or as provided on the engaging section of the first locking member may engage with a respective counter stop as provided on or in the body of the workpiece carrier. This way the movement of the first locking member towards and into the unlocking position can be stopped and/or defined.

According to a further example the first locking member is mechanically coupled with a spring element or with a respective restoring element configured to keep the first locking member in the unlocking position. By way of the spring or return element the first locking member can be permanently kept in the unlocking position provided that the receiving space is not occupied by a device component. By keeping the first locking member in the unlocking position unhindered insertion of the device component into the receiving space is inherently provided.

As soon as the device component engages the receiving section of the first locking member a respective movement of the first locking member towards and into the locking position is achieved, which movement acts against the return force of the spring element or return element. Typically, a gravitational force emanating from the device component upon insertion into the receiving space is larger than the force provided by the spring element. In this way, it is only or predominately by a gravitational effect of the device component that the first locking member is transferable into the locking position, thereby engaging with engaging section with the device component.

With other examples the workpiece carrier may be provided with a spring element. Here, the pivoting motion of the first locking member may be configured such, that the center of gravity of the first locking member is arranged or located relative to the hinge axis in such a way, that the first locking member tends to return into the unlocking position if the device component is not located inside the receiving space.

Also here, it may be the gravitational effect or gravitational force emanating from the device component when inserted into the receiving space that the first locking member is transferred and kept in the locking position, thereby fastening or holding the device component inside the receiving space at least with regard to the radial direction.

According to a further example the engaging section of the first locking member comprises a radially inwardly extending projection to engage with a radial recess of the device component. The radial recess of the device component provides a counter engaging portion or counter contact portion of the device component. When reaching a final assembly configuration or final assembly position inside the receiving space the radial recess of the device component longitudinally aligns with the engaging section of the first locking member.

By way of transferring or moving the engaging section radially inwardly towards a locking position the radially inwardly extending projection of the engaging section enters into the radial recess, thereby providing a well-defined radial fixing and holding of the device component relative to the body of the workpiece carrier. By the radially inwardly extending projection engaging with the radial recess of the device component there can be further provided a kind of a rotational interlock. When the circumferential width of the radial recess matches with the circumferential extension of the radially inwardly extending projection of the engaging section the device component is hindered to rotate with regard to the longitudinal direction as an axis of rotation. In this way there can be provided a well-defined orientation for the device component when received in the receiving space of the workpiece carrier.

According to a further example the projection of the engaging section comprises a first edge section and a second edge section separated along a circumferential direction with regards to the geometry of the tubular shaped receiving space. The first edge section is configured to engage with a first side edge of the radial recess. The second edge section is configured to engage with a second side edge of the radial recess of the device component. With some examples the radial recess of the device component is configured as a through opening extending through a sidewall of the device component. The radial recess may be provided by a window portion of a housing component of the drug delivery device.

With some examples the device component to be securely mounted to the workpiece carrier is implemented as a housing component of the respective drug delivery device. When the first and second edge sections comprise a circumferential distance that matches with the respective circumferential inside width of the recess of the device component a rather well-defined fixing and holding of the device component inside the receiving space can be provided. When the engaging section with its projection radially inwardly engages the radial recess of the device component the first edge section engages the first side edge of the radial recess and the second edge section engages the second side edge of the radial recess, thereby preventing any further rotational movement of the device component relative to the body of the workpiece carrier.

According to a further example the first edge section and the second edge section each comprise a first corner section and a second corner section radially and circumferentially offset from each other. In this way, the first side edge section and the second side edge section may each comprise a stepped profile. Each corner section may comprise a front face and a side face. A surface normal of the front face may face radially inwardly. A surface normal of the side face adjoining the front face may extend in tangential or circumferential direction. When appropriately engaging with a radial recess of the device component the front face of the corner section may engage a radially outwardly facing edge section of the side edge of the radial recess. A side face of a corner section adjoining the respective front face may engage or abut with a circumferentially facing edge portion of the side edge of the radial recess of the device component.

In this way, there can be provided a positive interlock as the projection of the engaging section enters radially inwardly into the radial recess of the device component. When the front face of the corner section gets in abutment with a radially outwardly facing edge portion of the device component the engaging section is hindered to move further radially inwardly into the radial recess. In this way, there can be provided a well-defined radially directed stop for the radially inwardly directed movement of the engaging section upon reaching the final assembly position.

By providing a first corner section and a second corner section the projection of the engaging section of the first locking member can be suitably adapted to varying geometries of differently sized radial recesses of device components of different types, e.g. of device components of different drug delivery devices.

With further examples, the first edge section and the second edge section of the projection of the engaging section of the first locking member may comprise a somewhat beveled or conical shape. They may be tapered radially inwardly. Hence, the circumferential width of the oppositely located edge sections of the projection of the engaging section reduces towards the radially inwardly facing direction. In this way, there can be provided a kind of a universal first and second edge section that is suitable to engage with radial recesses of the device component of different circumferential or tangential size.

According to a further example the locking mechanism comprises a second locking member functionally identical or functionally equivalent to the first locking member. Also the second locking member is movable relative to the body between a locking position and an unlocking position. The first locking member and the second locking member are located on opposite sides of the receiving space.

With some examples the first locking member and the second locking member are identically shaped. They are located at diametrically opposite positions on the sidewall of the body. They may both face radially inwardly so as to engage with oppositely located outside facing portions of the device component when the device component is longitudinally inserted into the receiving space. Insofar, all features, effects and benefits as described above with regards to the first locking member equally apply to the second locking member.

By having first and second locking members, there can be provided a twofold holding or fixing of the device component inside or relative to the body of the workpiece carrier. Upon insertion of the device component into the receiving space oppositely located outside facing portions of the device component may simultaneously engage with the receiving section of the first and the second locking member, thereby inducing a rather simultaneous movement of first and second locking members towards and into the locking position.

According to a further example of the workpiece carrier a longitudinal proximal end of an inside face of the engaging section comprises a beveled edge. The beveled edge faces radially inwardly. It extends radially inwardly as seen towards the distal direction. In this way, inserting of the device component into the receiving space is facilitated because the beveled edge provides a kind of a lead-in chamfer.

According to a further example a longitudinal proximal end of the engaging section comprises a protrusion extending obliquely in proximal direction radially outwardly. The protrusion may be arranged and configured as a longitudinal extension of the beveled edge of the engaging section. By way of a radially outwardly and proximally extending protrusion extending from the proximal end of the engaging section the extension of the beveled edge can be prolonged in proximal direction and radially outwardly. In this way, oppositely located beveled edges and radially outwardly extending protrusion at the proximal end of first and second locking members may comprise or form a kind of an insert funnel structure by way of which insertion of the device component into the receiving space located between the first and second locking members can be facilitated.

Moreover, by way of the beveled edge and/or by way of the obliquely extending protrusions at the proximal end of e.g. first and second locking members it is possible to separate the proximal end sections of the first and the second locking members away from each other in radial direction upon insertion of the device component into the receiving space. By way of a beveled edge or by way of obliquely extending protrusion at or near the proximal end of the first and the second locking member the first and second locking members may automatically return into the locking position upon removal of the device component from the receiving space. It is then by a repeated insertion of the device component into the receiving space, that the engaging sections of the first and/or second locking member are urged radially outwardly as the distal end of the device component slides along the beveled edge, thereby at least temporally switching or turning the first and/or second locking members into the unlocking position thereby enabling and supporting a rather smooth longitudinal insertion of the device component into the workpiece carrier.

With such examples it is no longer required to provide a spring element to the first locking member or to the second locking member. With suitably beveled sections or chamfered portions on the inside facing section at or near the proximal end of the first and/or the second locking members the workpiece carrier may be implemented or provided without such spring or return elements.

According to a further example, wherein the workpiece carrier comprises first and second locking members a radial distance between the first contact section of the first locking member and the second locking member when the first locking member and the second locking member are in the locking position is defined by a diameter or radial extent of a first device component. This way and when reaching a final assembly configuration inside the receiving space oppositely located and radially outwardly facing counter contact faces or counter contact portions of the first device component are in mechanical contact with the first contact section of the first locking member and the second locking member.

Here, the radial extent of the first device component urges the first and second locking members into the locking position. This movement may be delimited by a stop provided on the body to engage with a respective counter stop as provided e.g. on an outside face of the first and second locking member.

According to a further example and when the first and second locking member each comprise not only a first but also a second contact section a radial distance between the second contact sections of the first locking member and the second locking member when the first locking member and the second locking member are in the locking position is defined by a diameter or radial extent of a second device component. The shape and position of the second contact sections of first and second locking members is or are adapted to the shape, diameter or radial extent of respective counter contact sections of the second device component.

In this way and when provided with first and second contact sections being separated in longitudinal and radial direction from each other the first and second locking members can be universally used to lock a first device component or a second device component, wherein the first and the second device components distinguish by their geometric shape.

In a further aspect the present disclosure relates to a method of holding a device component of a drug delivery device in a production process, in particular in an automated production line by using a workpiece carrier as described above. Typically, the workpiece carrier may be implemented as an assembly nest configured to receive a subassembly of a drug delivery device, e.g. in an upright orientation. The method of holding the device component comprises the steps of inserting the device component in a longitudinal distal direction into the receiving space of the workpiece carrier, as a further step an outside facing first portion of the device component is brought in engagement with the receiving section of the first locking member, thereby inducing a movement of the first locking member into the locking position. When reaching the locking position there is established an engagement of the engaging section of the first locking member with a respective counter engaging section of the device component. Typically, the counter engaging section of the device component comprises a radial recess, e.g. provided in a sidewall of the respective device component.

For removing the device component out of the receiving space it may be sufficient to apply a proximally directed removal force onto the device component, thereby inducing a proximally directed movement of the device component relative to the body of the workpiece carrier. By such a removing in proximal direction the device component may get out of engagement with the receiving section, thereby allowing the first and/or the second locking members to return into the unlocking position. With some examples the counter engaging section of the device component, e.g. the radial recess of the device component engaging with the engaging section of the first and/or second locking member may engage with a beveled edge of the radially inwardly extending projection as provided on the engaging section of the first and/or the second locking members, thereby inducing a respective movement of the engaging sections towards the unlocking position.

Typically, the method of holding a device component is to be conducted and implemented by using a workpiece carrier as described above. Therefore, all features, effects and benefits as described above in connection with the workpiece carrier equally apply to the method of holding the device component on or in the workpiece carrier.

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE DRAWINGS

In the following, numerous examples of a data logging device for monitoring use of an injection device as well as a respective injection device will be described in greater detail by making reference to the drawings, in which:

FIG. 1 schematically illustrates a workpiece carrier with a device component assembled therein,

FIG. 2 shows an example of the workpiece carrier in a perspective illustration,

FIG. 3 is a top view of the workpiece carrier according to FIG. 2,

FIG. 4 shows a longitudinal cross-section through the workpiece carrier,

FIG. 5 shows first and second locking members in engagement with the device component,

FIG. 6 is an enlarged sectional view of FIG. 5,

FIG. 7 schematically shows a longitudinal cross-section through the workpiece carrier with a first device component assembled therein,

FIG. 8 shows a further example of the workpiece carrier holding another device component,

FIG. 9 is a cross-section through the engaging section of the workpiece carrier according to FIGS. 7,

FIG. 10 is a cross-section through the engaging section of the workpiece carrier according to FIG. 8,

FIG. 11 schematically shows an example of a drug delivery device,

FIG. 12 is a flowchart of a method of holding a device component,

FIG. 13 is a perspective illustration of a further example of a workpiece carrier,

FIG. 14 shows an arrangement of a first device component held by first and second locking members of the workpiece carrier according to FIG. 13 and

FIG. 15 shows an arrangement of a second device component engaged with first and second locking members of the workpiece carrier according to FIG. 13.

### DETAILED DESCRIPTION

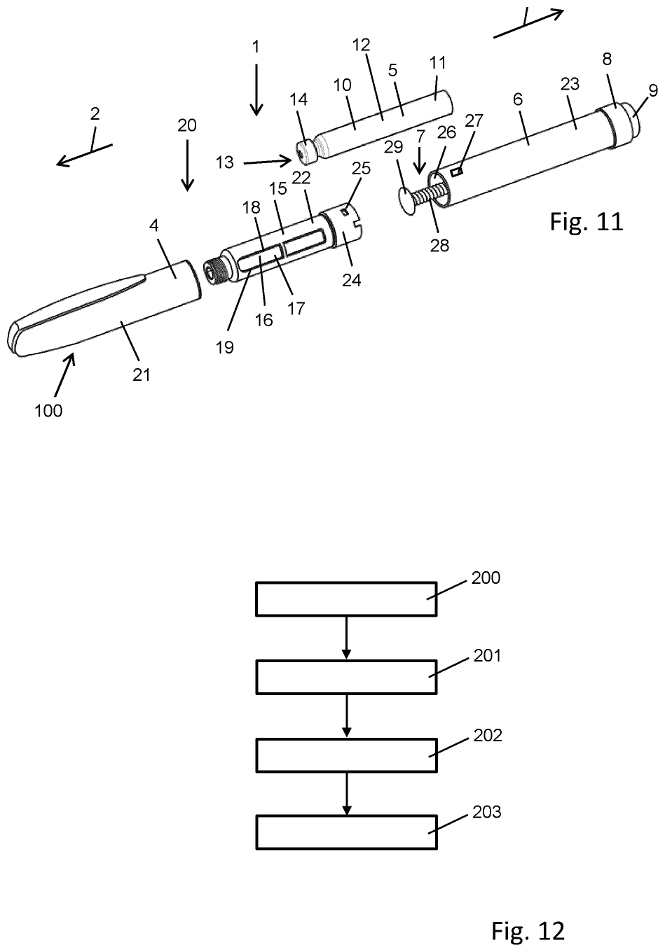

In FIG. 11 there is illustrated one example of a drug delivery device 1, which is implemented as a handheld injection device or as a pen-type injector. Typically, the drug delivery device 1 comprises an elongated shape. It may extend along a longitudinal direction. Towards a longitudinal distal direction 2 the drug delivery device 1 comprises an outlet opening for dispensing a medicament. Towards a proximal end 3 the drug delivery device 1 comprises at least one of a dose member 8 and a trigger 9, by way of which a dose of equal or individual and different size can be set and dispensed, respectively. The drug delivery device 1 comprises a housing 20. The housing 20 as well as the drug delivery device 1 may comprise numerous device components 100. For instance, the drug delivery device 1 may comprise a protective cap 4 covering a distal housing component configured as a cartridge holder 15.

The housing 20 may further comprise a body 6, e.g. forming a proximal end of the housing 20. The body 6 is configured to house a drive mechanism 7. Typically, the drive mechanism 7 comprises a piston rod 28, e.g. provided with a pressure piece 29 located at its distal end. The cartridge holder 15 is sized and configured to accommodate a medicament container 5. In the present example the medicament container 5 comprises a cartridge 10. The cartridge 10 comprises a tubular-shaped barrel 11 containing the liquid medicament 12. Towards a distal end the cartridge 10 comprises a dispensing end 13, which may be closed and covered by a seal 14. The seal 14 may be pierceable by a double-tipped injection needle. With some examples the injection needle (not illustrated) is connectable to a distal end of the cartridge holder 15. The distal end of the cartridge holder may comprise a threaded socket to engage with a complementary shaped part of the needle assembly.

The distal end of the cartridge holder 15 may comprise a through opening to receive a distally pointing portion of the double-tipped injection needle configured to pierce and to penetrate the seal 14 of the cartridge 10. With other examples (not illustrated) the cartridge 10 or medicament container 5 may be readily equipped with an injection needle. Typically, a proximal end of the medicament container 5 is sealed by a movable stopper. The stopper is movable along the longitudinal direction inside the barrel under the action of the advancing piston rod 28 thereby expelling a dose of the medicament 12 from the medicament container 5.

As particularly illustrated in FIG. 11 the cartridge holder 15, hence a sidewall 22 of the cartridge holder 15 comprises a recess 16 implemented as an aperture 17 and hence as a through opening extending through the sidewall 22. As further illustrated the recess 16 comprises longitudinally extending oppositely located side edges 18, 19 separated from each other along a circumferential direction of the tubular or elongated sidewall 22 of the cartridge holder 15.

A proximal end of the cartridge holder 15 is implemented as a proximal connecting end configured to engage with a distal connecting end 26 of the body 6. This way, the proximally located connecting end 24 of the cartridge holder 15 can be connected to a distally located connecting end 26 of the body 6. The connecting end 24 is provided with a fastening element 25 complementary shaped to a counter fastening element 27 of the body 6. With some examples the proximal connecting end 24 of the cartridge holder 15 comprises a radially stepped down insert configured for longitudinal insertion into a distally located receptacle formed by the sidewall 23 of the body 6.

The presently illustrated example of a drug delivery device 1 is only exemplary for a large variety of injection devices 1 that could be manufactured in a mass manufacturing process and hence in an automated production line. The drug delivery device one may comprise a fixed dose injection device, a variable dose injection device, an autoinjector, and all-mechanically implemented pen-type injector, with or without a movable dial extension or an electronically or semi-electronically implemented injection pen.

With some examples a cartridge 10 or medicament container 5 readily inserted in the cartridge holder 15 may form or constitute a first subassembly. The drive mechanism 7 mounted inside the body 6 may form a second subassembly. During manufacturing the first and second subassemblies may require a precise mutual assembly. For this it may be required that one of the first and second subassemblies is held in an upright orientation by way of a workpiece carrier 30 or nest assembly as further described below.

Moreover, it is conceivable that dedicated cartridges 10 or devices 1, hence any of the above illustrated individual device component 100 requires an automated semi-automated handling in an automated production line. Insofar, any of the illustrated housing components, e.g. the protective cap 4, the cartridge holder 15, the cartridge 10 or the body 6 may represent a device component 100 intended for insertion into a workpiece carrier 30 as described further below. With some examples the device component 100, e.g. in form of the protective cap 4, the cartridge holder 15 or the body 6 or combinations thereof may have to be placed and oriented in a vertical configuration inside a workpiece carrier 30, e.g. for further steps of device assembly and/or for attaching e.g. a label to an outside surface of the respective device component 100, e.g. to an outside surface of any of the illustrated sidewalls 21, 22, 23.

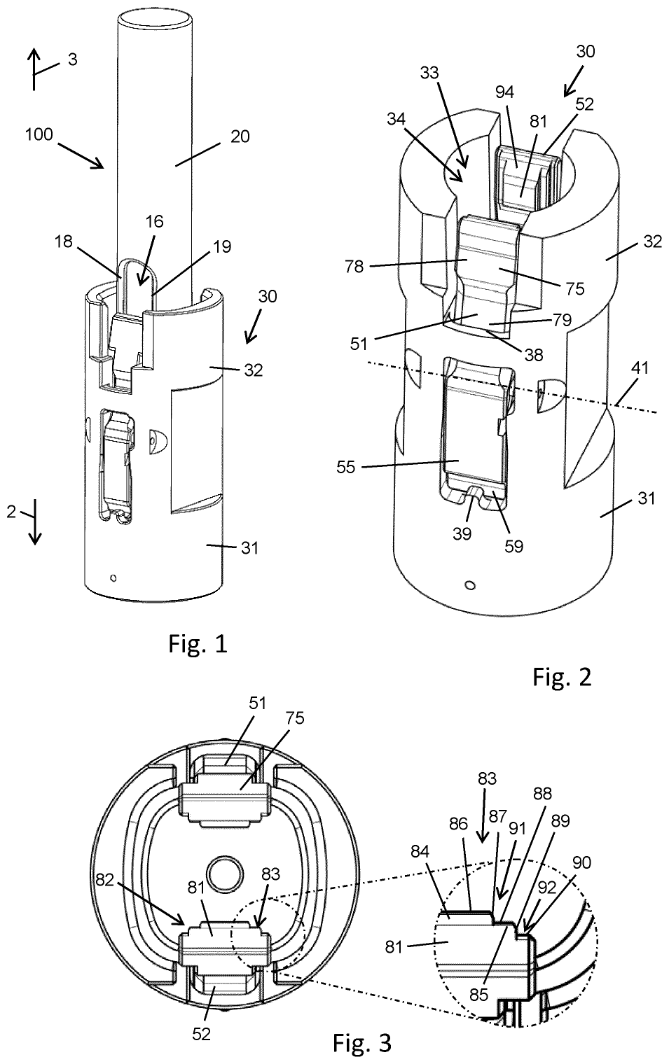

In FIGS. 1-10 there is illustrated a first example of a workpiece carrier 30, e.g. implemented as an assembly nest. The workpiece carrier 30 comprises a body 31. The body 31 is of substantially tubular shape. The body 31 comprises a tubular-shaped sidewall 33 defining a respective tubular shaped and elongated receiving space 33.

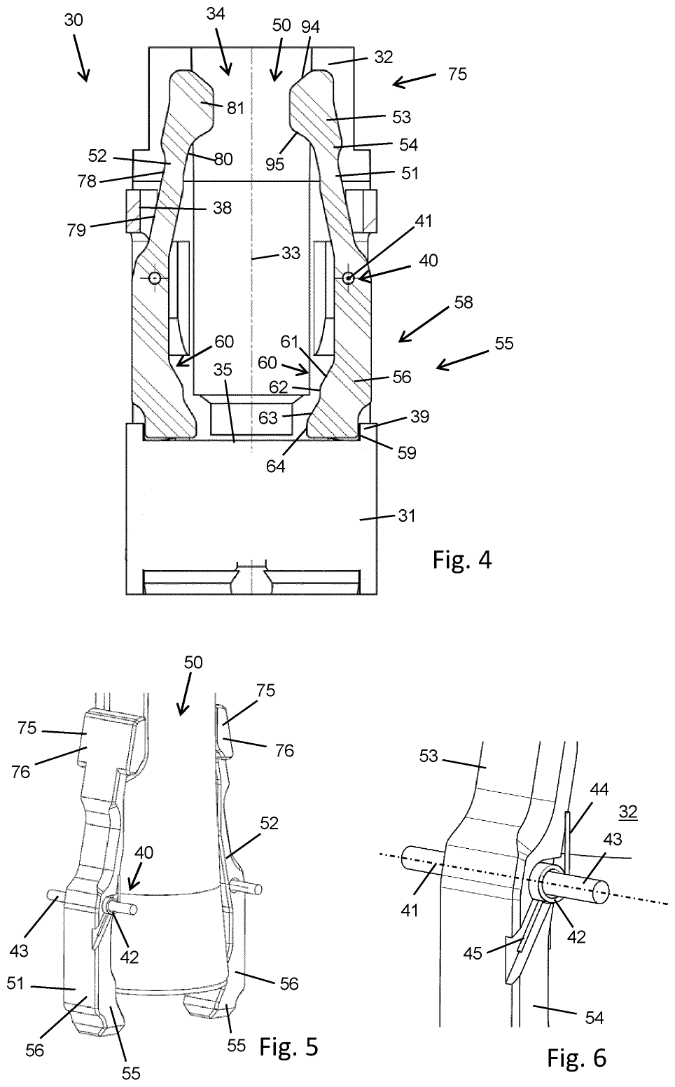

Towards an upper or towards a proximal end the sidewall 32 defines an insert opening 34, through which a device component 100 can be inserted into the receiving space 33 in longitudinal direction, e.g. in a longitudinal distal direction 2. As illustrated in FIG. 4, the receiving space 33 is delimited in distal direction 2 by a bottom 35.

The workpiece carrier 30 further comprises a locking mechanism 50 particularly implemented to fix and/or to hold the device component 100 inside the receiving space 33, once the device component 100 has reached a final assembly or final holding position inside the receiving space 33.

The locking mechanism 50 comprises a first locking member 51 and a second locking member 52. Each locking member 51, 52 comprises an elongated locking arm 53. Each locking arm 53 extends along the longitudinal direction. The locking members 51, 52 with their respective locking arms 53 are movably disposed on the body 31. They are rotationally or pivotally supported on or in the sidewall 32 of the body 31. As shown in FIGS. 4-6 the locking members 51, 52 are mounted and supported on or in the sidewall 32 by way of a hinge 40. The hinge 40 comprises a hinge axis 41. The hinge axis 41 substantially extends in tangential direction with regards to the tubular shape of the sidewall 32. The hinge 41 comprises an elongated shaft 43 extending through a respective receptacle or bore of the locking members 51, 52. As further illustrated in FIGS. 5 and 6 the hinge 40 is provided with a spring element 42. The spring element 42 is implemented as a torsion spring.

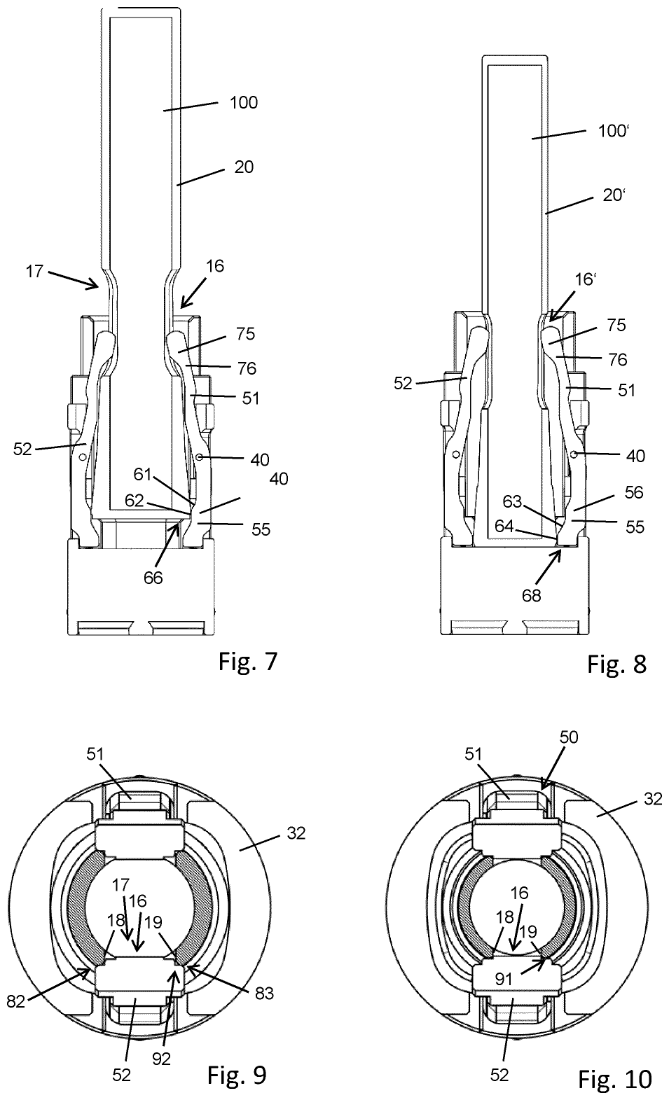

The spring element 42 comprises a first leg 44 and a second leg 45. The first leg and 44 is in engagement or abutment with the body 31, in particular with the sidewall 32, whereas the second leg 45 is in engagement or abutment with the locking arm 53. In this way, the respective locking arm 53 and hence the first and/or the second locking members 51, 52 are movable against the action of the spring element 42. With some examples the locking members 51, 52 are movable relative to the body 31 between a locking position, e.g. as illustrated in FIGS. 7 and 8 and an unlocking position.

Typically, the spring element 42 may be operable to keep the locking members 51, 52 in the locking position, in which a transverse size or inner cross-section of the insert opening 34 is at a maximum. By way of the spring element 42, the locking mechanism 50 can be automatically transferred into the locking position, especially, when the device component 100 has been removed from the receiving space 32. Hence, in the unlocking position the proximal section 75 or proximal end of the locking members 51, 52 is at a radially outwardly directed end position. When in the locking position the locking members 51, 52 are pivoted radially inwardly with the proximal section 75 so as to engage with an outside surface of the device component 100.

In the following, the structure of the locking member 51, 52 is described in more detail. The locking members 51, 52 comprise an elongated body 54. The body 54 may be of substantially oblong shape. The locking members 51, 52, hence the locking arm 53 and the body 54 thereof comprise a distal section 55, which is located on one side, e.g. on a lower side of the hinge 40. The locking members 51, 52 further comprise a proximal section 75, which is the part of the locking members 51, 52 protruding upwardly and hence proximally from the hinge 40.

Due to the hinged engagement with the body 31 a radially outwardly directed displacement of the distal section 55 leads to a radially inwardly directed movement of the proximal section 75. The distal section 55 comprises a receiving section 56 at an inside face 60. The inside face 60 faces towards the receiving space 33, which is sized and shaped to receive the device component 100. Likewise, the proximal section 75 comprises an engaging section 76 on its inside face 80. The engaging section 76 comprises a radially inwardly extending projection 81. The projection 81 is sized and configured to make contact with an outside surface of the device component 100, e.g. with an outside surface of a housing 20 or housing component, such as a sidewall 21, 22, 23 as described above. With some examples and when the device component 100 comprises a recess 16 the radially inwardly extending projections 81 of the engaging sections 76 are configured to extend into the recess 16 as for instance illustrated in FIGS. 9 and 10.

The receiving section 56, e.g. provided at the distal end of the locking arm 53 comprises a first beveled section 61 followed by a first longitudinally shaped contact section 62. The first contact section 62 is followed in distal direction 2 by another, hence by a second beveled section 63, which further extends into another, hence into a second contact section 64 as provided at the very distal end of the inside face 60 of the locking arm 53.

The locking arm 52 of the second locking member 52 is implemented identical to the locking arm 53 of the first locking member 51. The first and second locking members 51, 52 are located on opposite sides of the receiving space 33.

As further illustrated in FIG. 4 the locking members 51, 52 comprise an outer stop 59 or outer stop face on an outside face 58 to engage with a counter stop 39 as provided at or near the bottom 53. In this way, a counterclockwise rotational or pivoting motion of the first locking member 51 as illustrated in FIG. 4 can be delimited and stopped. When the outer stop 59 of the first locking member 51 engages radially with the counter stop 39, the locking position has reached and the receiving section 56 cannot be displaced further in radial outer direction.

Also the proximal section 75 is provided with a respective counter stop 38. Here, the counter stop 38 is located above, hence at a longitudinal proximal offset from the hinge 40. The counter stop 38 is located radially outwardly from an outside face 78 of the proximal section 75. In the locking position as illustrated in FIG. 4, an outer stop 79 of the outside face 78 of the proximal section 75 is located at a radial distance from an inside face of the counter stop 38. However, if for instance the first locking member 51 is rotated in clockwise direction. FIG. 4, e.g. induced by the spring element 42, the counter stop 38 limits such a pivoting motion and keeps the respective locking member 51 in the unlocking position. As further illustrated in FIG. 4, the outside faces 78 of the locking members 51, 52 each comprise an outer stop 79, which gets in abutment with an inside facing portion of the counter stop 38 when reaching the unlocking position.

The a stepped profile at or near the distal end on the inside face 60 of the first and second locking members 51, 52 is beneficial to provide a universal assembly nest or workpiece carrier 30 suitable for insertion and securely holding different types of device components 100.

In the illustration of FIG. 7 a first device component 100, e.g. a first housing 20 is inserted into the receiving space 30 in distal direction 2. The distal end of the device component 100 comprises a first radial extent, that matches with the radial distance between oppositely located first contact sections 62 of the first and the second locking members 51, 52. The first beveled section 61 located proximally offset and adjoining in proximal direction the first contact section 61 provides a kind of a guiding or sliding surface during the insert motion of the device component 100.

Before inserting the device component 100 into the receiving space 33 the first and second locking member 51, 52 are in the unlocking position. Compared to the illustration of FIG. 7 the radial distance between the distal section 55 of the first and the second locking members 51, 52 is somewhat smaller than illustrated. Concurrently, the radial distance between the proximal sections 75 and hence of the engaging sections 76 of the first and second locking members 51, 52 is larger in the unlocking position than illustrated in FIG. 7. This allows and provides an unhindered insertion of the device component 100 through the insert opening 34 into the receiving space 33.

As the distal end or distal edge of the device component 100 engages with the first beveled sections 61 of the first and the second locking member 51, 52 there is induced a radially outwardly directed pivoting motion of the respective receiving sections 56 of the first and the second locking members 51, 52 and is the distal edge or distal end of the device component 100 that slides along the first beveled section until reaching or engaging the contact sections 62.

Since the contact sections 62 are delimited in distal direction by a further radially inwardly extending beveled section 63 which in the present configuration further constricts the free space inside the body 31, the transition from the first contact section 62 into the second beveled section 63 provides a first longitudinal end stop 66 for the insert motion of the device component 100 into the receiving space 33.

With the further example of FIG. 8 the further device component 100′ is of slightly different shape. Here, the distal end of the device component 100′ has a slightly smaller radial extent compared to the device component 100 as illustrated in FIG. 7. Accordingly, the device component 100′ is sized to squeeze between the second contact sections 63 of the oppositely located locking members 51, 52 when reaching a distal and hence a final assembly configuration. Here, the closed bottom 35 of the body 31 forms a further end stop 68 to prevent any further insert motion in distal direction 2 of the device component 100.

Since the device component 100 serves to urge the receiving section 56 of the first and the second locking member 51, 52 radially outwardly the engaging section 76 of the respective first and second locking members 51, 52 will rotate or move radially inwardly so as to engage with the recesses 16 or apertures 17 that align with the projections 81 of the first and second locking member 51, 52. This way, a kind of a radially inwardly directed clamping effect can be applied to the device component 100 when reaching the final assembly or final holding configuration. With both configurations as illustrated in FIGS. 7 and 8 the device component 100 is fixed and stabilized in a twofold manner inside the receiving space 33. The distal end section of the device component is squeezed or sandwiched between oppositely facing contact sections 62, 64 at or near the distal end of the respective locking members 51, 52.

A further portion, hence a counter contact portion of e.g. provided by the recesses 16 or apertures 17 is in radial engagement with the radially inwardly pivoted engaging sections 76 of the first and second locking members 51, 52.

As it is further illustrated in the combination of FIGS. 3, 9 and 10 the projection 81 as provided at the proximal section 75 of the locking members 51, 52 comprises a first edge section 82 and a second edge section 83. The first and second edge sections 82, 83 are separated in circumferential or tangential direction with regards to the elongated shape or tubular shape of the body 31. As particularly illustrated in FIGS. 9 and 10 the first edge section 82 is configured to engage a first longitudinal side edge 18 of a recess 16 or aperture 17 of the device component 100. The oppositely located second edge section 83 is configured to engage with the oppositely disposed second side edge 19 of the recess 16 or aperture 17.

As shown in greater detail in FIG. 3 the first and second edge sections 82, 83 comprises two corner section 91, 92 forming a stepped profile of the radially inwardly facing first and second edges 82, 83. Hence, the radially inwardly facing portion of the projection 81 comprises a first step section 84 and a second step section 85 that distinguish by their circumferential extend and radial position.

The radially inwardly located step section 84 is delimited in circumferential direction by a side face 87 extending in radial direction and thus faxing in circumferential direction. The side face 87 merges into a front face 88 facing radially inwardly. The front face 88 and the side face 87 form or constitute a first corner section 89. An opposite circumferentially outside facing portion or end of the front face 88 merges into another side face 89 extending in radial direction. The side face 89 merges further into another front face 90 facing radially inwardly. The side face 89 and the front face 19 form or constitute a second corner section 92.

When engaging with the recess 16 or aperture the side faces 87, 89 engage with a circumferentially facing edge portions of the side edges 18, 19 of the recess 16 or aperture 17. The front faces 88, 89 serve to abut radially inwardly against an outside facing edge portion of the side edges 18, 19 as illustrated in FIGS. 9 and 10.

The stepped profile of the inside face 80 of the projection 81 is particularly shaped and adapted to match with differently sized apertures 17 or recesses 16 provided on differently-shaped device components 100, 100′.

Accordingly and with the example of a device component 100 as illustrated in FIGS. 7 and 9 it is the second corner section 92 that engages a respective outwardly facing edge portion of the side edges 18, 19 of the aperture 17. With the further example of FIGS. 8 and 10, wherein another device component 100′ is inserted into the receiving space 33 it is the first corner section 91 that engages with the respective side edges 18, 19 of the recess 16. In this way, the inside face 80 of the projection 81 matches in size and shape with the recesses 16 as provided on the outside surface of a sidewall of the device component 100 in order to provide a rather stable and tilt free arrangement and holding of the device component 100 on or in the workpiece carrier 30.

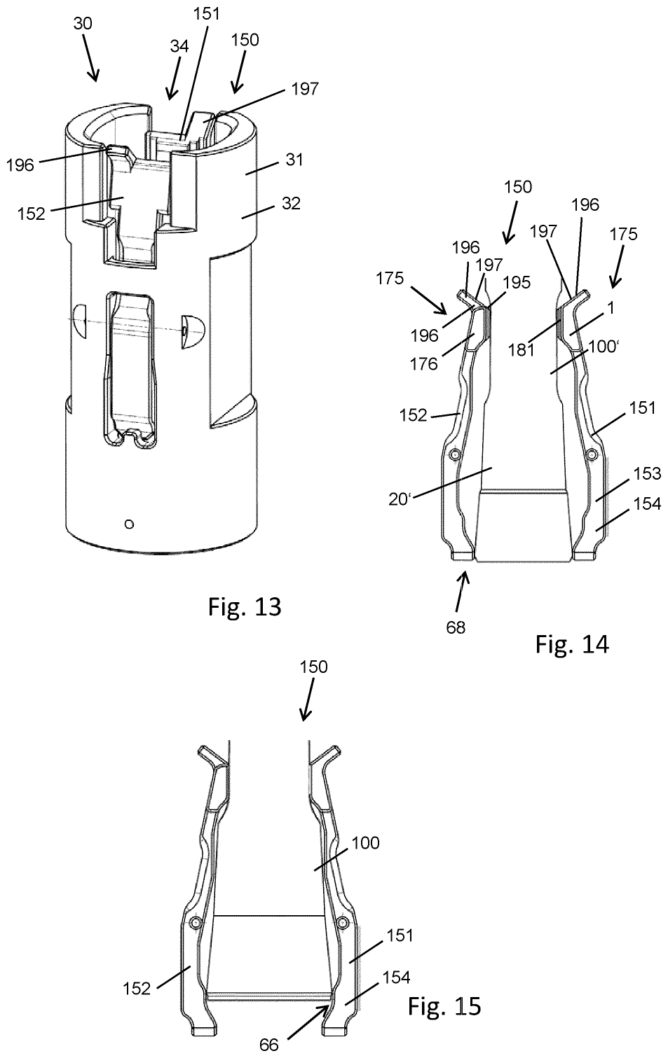

In the further example as illustrated in FIGS. 13-15 there is shown a further example of another locking mechanism 150. This locking mechanism 150 also comprises first and second locking members 151, 152 both implemented as a locking arm 153 comprising an elongated body 154. The only difference of the locking members 151, 152 compared to the locking members 51, 52 is provided at the proximal end thereof. There, the proximal section 175 also comprises an engaging section 176 with an inside face 180 provided with a projection 181.

The longitudinal proximal end of the engaging section 176 comprises a radially outwardly and proximally extending protrusion 196 extending obliquely in proximal direction 3 and radially outwardly. A proximal end face comprises a gliding surface 197 forms a chamfered section 195. This obliquely extending protrusion 196 serves to engage with a distal end of the device component 100 upon inserting the device component 100 into the receiving space 33. When engaging with the gliding surface 197 of the chamfered section 195 insertion of the device component 100 into the receiving space 33 in distal direction 2 induces a radially outwardly directed movement of the locking members 151, 152 towards and into the unlocking position thus increasing the free space provided between the proximal sections 175 of the oppositely located locking members 151, 151 to facilitate a smooth insert motion of the device component 100 into the interior of the body 31.

Again and when reaching the distal final assembly position as illustrated in FIGS. 14 and 15 the distal end section of the device component 100 engages the receiving sections 156, thereby urging the preceding sections 156 away from each other so that the engaging sections 176 move radially inwardly to clamp or to engage with e.g. the recesses 16 provided on the outside surface of the housing component 100, 100′.

In the flowchart of FIG. 12 the method of holding a device component with 100 making use of a workpiece carrier 30 as described herein is described. In a first step 200 the device component 100 is inserted in longitudinal direction into the receiving space 33. Thereby, and in step 201 an outside facing first portion of the device component 100, e.g. a distal end section of the device component 100 gets in engagement with the receiving section 56 of the first locking member 51. Thereby and in step 202 there is induced a movement of the first locking member into the locking position. In a further step 203 and concurrently with the movement of the first locking member induced by the insert motion of the device component 100 an engagement of the engaging section 76 with a second portion, e.g. with a radial recess 16 of the device component 100 is established in step.

With the example of FIGS. 13-15 it is not necessary that the first and second locking members 51, 52 are kept in the unlocking position when the device component 100 has been removed from the receiving space 33. Depending on the geometry and/or depending on the center of gravity of the pivotable locking members 51, 52 they may assume their locking position automatically upon removal of the device component 100.

By way of the chamfered section 195 and the radially outwardly extending protrusion 196 there will be induced a smooth movement or transfer of the locking members 151, 152 towards the unlocking position during insertion of the device component 100 into the receiving space 33.

With any of the presently illustrated examples of the workpiece carriers 30, the engaging section 76 may comprise a distally facing the beveled edge 95 on the inside face 80 of the radially inwardly extending projection 81. this beveled edge 95 may help to induce a pivoting motion of the locking members 51, 52, 151, 152 from the locking position towards and into the unlocking position, namely as the device component 100 is subject to a movement in proximal direction 3 relative to the body 31. Then, a distal edge of the recesses 16 may engage the beveled section or beveled edge 95 thereby inducing a respective radially outwardly directed movement of the engaging section 76 and hence of the proximal end 75 of the locking members 51, 52, 151, 152.

### REFERENCE NUMBERS

1 drug delivery device
2 distal direction
3 proximal direction
4 protective cap
5 medicament container
6 body
7 drive mechanism
8 dose member
9 trigger
10 cartridge
11 barrel
12 medicament
13 dispensing end
14 seal
15 cartridge holder
16 recess
17 aperture
18 side edge
19 side edge
20 housing
21 sidewall
22 sidewall
23 sidewall
24 connecting end
25 fastening element
26 connecting end
27 fastening element
28 piston rod
29 pressure piece
30 assembly nest
31 body
32 sidewall
33 receiving space
34 insert opening
35 bottom
38 counter stop
39 counter stop
40 hinge
41 hinge axis
42 spring
43 shaft
44 leg
45 leg
50 locking mechanism
51 locking member
52 locking member
53 locking arm
54 body
55 distal section
56 receiving section
58 outside face
59 outer stop
60 inside face
61 beveled section
62 contact section
63 beveled section
64 contact section
66 end stop
68 end stop
75 proximal section
76 engaging section
78 outside face
79 outer stop
80 inside face
81 projection
82 edge section
83 edge section
84 step section
85 step section
86 front face
87 side face
88 front face
89 side face
90 front face
91 corner section
92 corner section
94 beveled edge
95 beveled edge
100 device component
150 locking mechanism
151 locking member
152 locking member
153 locking arm
154 body
156 receiving section
175 proximal section
176 engaging section
180 inside face
181 projection
195 chamfered section
196 protrusion
197 gliding surface

1. A workpiece carrier (30) for holding a device component (100) of a drug delivery device (1) in an automated production line, the workpiece carrier (30) comprising:
a body (31) extending in a longitudinal direction (z), the body (31) defining a receiving space (33) sized to receive at least a portion of the device component (100),
a locking mechanism (50; 150) comprising a first locking member (51; 151) movable relative to the body (31) between a locking position and an unlocking position,
wherein the first locking member (51; 151) comprises a receiving section (56) and an engaging section (76; 176) separated from the receiving section (56),
wherein the receiving section (56) adjoining the receiving space (33) comprises an inside face (60) with a first contact section (62) facing radially inwardly towards the receiving space (33) to make contact with the device component (100) upon insertion of the device component (100) into the receiving space (33) along the longitudinal direction (z) to thereby induce a movement of the first locking member (51; 151) into the locking position.

2. The workpiece carrier (30) according to claim 1, wherein the first locking member (51; 151 comprises a locking arm (53) pivotally attached to the body (31) between the locking position and the unlocking position.

3. The workpiece carrier (30) according to any one of the preceding claims, wherein the receiving section (56) and the engaging section (76; 176) of the first locking member (51; 151) are located on opposite sides of a hinge axis (41) such that a radially outwardly directed movement of the receiving section (56) inducible by insertion of the device component (100) into the receiving space (33) causes a radially inwardly directed movement of the engaging section (76; 176).

4. The workpiece carrier (30) according to any one of the preceding claims, wherein the first locking member (51; 151) extends in the longitudinal direction (z) and wherein the receiving section (56) and the engaging section (76; 176) are separated along the longitudinal direction (z).

5. The workpiece carrier (30) according to any one of the preceding claims, wherein the inside face (60) of the receiving section (56) comprises a first beveled section (61) adjoining longitudinally into the first contact section (62).

6. The workpiece carrier (30) according to any one of the preceding claims, wherein the inside face (60) comprises a second contact section (64) facing radially inwardly towards the receiving space (33) to make contact with the device component (100) upon insertion into the receiving space (33).

7. The workpiece carrier (30) according to claim 6, wherein the inside face (60) of the receiving section (56) comprises a second beveled section (63) adjoining longitudinally into the first contact section (62) and adjoining longitudinally into the second contact section (64).

8. The workpiece carrier (30) according to any one of the preceding claims, wherein the first locking member (51; 151) is mechanically coupled with a spring element (42) configured to keep the first locking member (51; 151) in the unlocking position.

9. The workpiece carrier (30) according to any one of the preceding claims, wherein the engaging section (76; 176) comprises a radially inwardly extending projection (81) to engage with a radial recess (16) of the device component (100).

10. The workpiece carrier (30) according to claim 9, wherein the projection (81) of the engaging section (76; 176) comprises a first edge section (82) and a second edge section (83) separated along a circumferential direction with regard to the geometry of the receiving space (33), wherein the first edge section (82) is configured to engage with a first side edge (18) of the radial recess (16) and wherein the second edge section (83) is configured to engage with a second side edge (19) of the radial recess (16) of the device component (100).

11. The workpiece carrier (30) according to claim 10, wherein the first edge section (82) and the second edge section (83) each comprise a first corner section (91) and a second corner section (92) radially and circumferentially offset from each other.

12. The workpiece carrier (30) according to any one of the preceding claims, wherein the locking mechanism (50; 150) comprises a second locking member (52; 152) functionally identical to the first locking member (51; 151) and movable relative to the body (31) between a locking position and an unlocking position, wherein the first locking member (51; 151) and the second locking member (52; 152) are located on opposite sides of the receiving space (33).

13. The workpiece carrier (30) according to any one of the preceding claims, wherein a longitudinal proximal end of an inside face (80) of the engaging section (76; 176) comprises a beveled edge (94).

14. The workpiece carrier (30) according to any one of the preceding claims, wherein a longitudinal proximal end of the engaging section (176) comprises a protrusion (196) extending obliquely in proximal direction and radially outwardly.

15. A method of holding a device component (100) of a drug delivery device (1) in an automated production line by using a workpiece carrier (30) as defined by any of the preceding claims, the method comprises the steps of:
inserting the device component (100) in a longitudinal distal direction (2) into the receiving space (33) of the workpiece carrier (30),
bringing an outside facing first portion of the device component (100) in engagement with the receiving section (56) of the first locking member (51; 151) and thereby
inducing a movement of the first locking member (51; 151) into the locking position and
establishing an engagement of the engaging section (76; 176) of the first locking member (51; 151) with a radial recess (16) of the device component (100).
